(19) United States
(12) Patent Application Publication
(10) Pub. No.: US 2021/0169691 A1
(43) Pub. Date: Jun. 10, 2021

(54) METHOD FOR DETERMINING A CURRENT POSITION OF A PATIENT INTERFACE OF AN EYE SURGICAL LASER BASED ON A PURKINJE IMAGE

(71) Applicant: Schwind eye-tech-solutions GmbH, Kleinostheim (DE)

(72) Inventors: Samuel Arba-Mosquera, Aschaffenburg (DE); Mario Shraiki, Stockstadt (DE); Nico Triefenbach, Mainaschaff (DE)

(21) Appl. No.: 17/112,319

(22) Filed: Dec. 4, 2020

(30) Foreign Application Priority Data
Dec. 6, 2019 (DE) 10 2019 133 431.4

(51) Int. Cl. A61F 9/008; A61B 3/10; A61B 3/14

(52) U.S. Cl. CPC A61F 9/0084; A61B 3/101; A61F 2009/00872; A61F 2009/00846; A61F 2009/00897; A61B 3/14

(57) ABSTRACT

A method is disclosed for determining a current position of a patient interface of an eye surgical laser for an eye relative to an optical axis of a laser beam of a treatment apparatus. The method includes determining a target position of the patient interface relative to the optical axis, positioning the patient interface in a preset area in front of the optical axis, illuminating the patient interface by means of an illumination device, capturing a Purkinje image by means of the optical capturing device, comparing the captured Purkinje image to the optical axis and determining the current position of the patient interface depending thereon, comparing the current position to the target position and with a deviation, and outputting a control signal to a control device of the treatment apparatus. A treatment apparatus, a computer program and a computer-readable medium are disclosed for carrying out the method.

The invention relates to a method for determining a current position of a patient interface of an eye surgical laser of a treatment apparatus for an eye of a patient relative to an optical axis of a laser beam of the laser in a neutral pose of a beam deflection device of the treatment apparatus. Further, the invention relates to a treatment apparatus, to a computer program and to a computer-readable medium.

Opacities and scars within the cornea, which can arise by inflammations, injuries or congenital diseases, impair the sight. In particular in case that these pathological and/or unnaturally altered areas of the cornea are located in the axis of vision of the eye, clear sight is considerably disturbed. Hereto, different laser methods by means of corresponding treatment apparatuses are given from the prior art, which can separate a volume body from the cornea and thus improve the sight for a patient. These laser methods are in particular an invasive intervention such that it is of particular advantage for the patient if the intervention is performed in a time as short as possible and to a particularly efficient extent. Therein, the volume body in particular is to only include the altered area of the cornea. Therefore, based on the prior art, it is particularly important to be able to perform an accurate position determination of the devices of the treatment apparatus, which are used in the intervention.

Therefore, it is the object of the present invention to provide a method and a treatment apparatus, by means of which a current position of a patient interface of the treatment apparatus can be captured in improved manner.

This object is solved by a method, a treatment apparatus, a computer program as well as a computer-readable medium according to the independent claims. Advantageous configurations with convenient developments of the invention are specified in the respective dependent claims, wherein advantageous configurations of the method are to be regarded as advantageous configurations of the treatment apparatus, of the computer program and of the computer-readable medium and vice versa.

An aspect of the invention relates to a method for determining a current position of a patient interface of an eye surgical laser of a treatment apparatus for an eye of a patient relative to an optical axis of a laser beam of the laser in a neutral pose of a beam deflection device of the treatment apparatus. Determining a target position of the patient interface relative to the optical axis is performed. The patient interface is positioned in a preset area in front of the optical axis. The patient interface is illuminated by means of an illumination device of the treatment apparatus. Capturing a Purkinje image associated with the patient interface is effected by means of an optical capturing device of the treatment apparatus. The captured Purkinje image is compared to the optical axis and the current position of the patient interface is determined depending thereon. Comparing the current position to the target position is effected, and with a deviation of the current position from the target position, a control signal is output to a control device of the treatment apparatus.

Thus, the current position of a patient interface with respect to the optical axis of the treatment apparatus, in particular with respect to the laser beam, can be determined in improved manner.

Thereto, the method according to the invention in particular exploits that the patient interface, which can also be referred to as contact element, is curved, but for example formed more flatly than a human cornea. Due to the curvature, a so-called Purkinje image arises upon the illumination. The Purkinje image in turn is associated with the patient interface. In particular, a determined position of the Purkinje image in the captured image can be used for comparison. Based on the Purkinje image, which is associated with the patient interface, thus, a position of the patient interface relative to the optical axis can be determined. In particular, the target position of the patient interface relative to the optical axis has already been previously determined. Based on the comparison of the determined position based on the Purkinje image to the target position of the patient interface, thus, a deviation can be determined. In particular, a corresponding control signal can then be output to a control device of the treatment apparatus depending on the deviation. According to the invention, the position of the patient interface can in particular be determined based on the Purkinje image. For example, a decentration, but also a tilting, of the patient interface with respect to the optical axis can be determined. Thus, an error source can be determined, whereby a decentration to the optical axis can for example be compensated for based on a corresponding control signal.

In particular, the beam deflection device has a neutral pose. For example, the beam deflection device can have two mirrors for deflecting the laser beam. Then, the neutral pose is given with a so-called 0/0 pose of the mirrors to each other. With a rotation of the mirrors, the incident laser beam experiences a deflection and thus can for example be positioned on the cornea. Thus, the beam deflection device has a rotational axis around which the incident laser beam can be rotated depending on the mirror positions. The optical axis in particular describes the position of the laser beam in the neutral pose of the beam deflection device, which can also be referred to as scanner.

According to an advantageous form of configuration, a first order or second order Purkinje reflex is captured as the Purkinje image. In particular, a Purkinje image of the first order Purkinje reflex is captured. Thereby, it is allowed that an automatic patient interface tracking can for example be performed by means of the treatment apparatus since the first order Purkinje reflex is in particular very well optically capturable by means of the capturing device. Thus, a manual intervention by a user, for example an optician, is not required to be able to perform a corresponding position correction.

Further, it has proven advantageous if with an ascertained deviation, a control signal is generated such that a position correction of the patient interface or of the optical axis is performed. In other words, if the determined current position should deviate from the target position, thus, either the patient interface can be positioned to get from the current position to the target position, or the optical axis can be corrected, for example a displacement of the beam deflection device. Thereby, it is allowed that a treatment of the patient can nevertheless be reliably performed even with an ascertained deviation.

Further, it has proven advantageous if after an ascertained deviation below a preset deviation threshold value, a control signal is generated such that a docking procedure of the patient interface to the eye is performed. For example, if a deviation should be ascertained, but it should be low, thus, a docking procedure to the eye can nevertheless be performed after determining the position. Thereby, the treatment time can be reduced.

It is also advantageous if the patient interface is illuminated by means of an illumination ring or illumination point or by means of an illumination half ring or by means of illumination sectors of the illumination device for generating the Purkinje image. In particular, it is allowed by the different forms of configuration that the Purkinje image can be reliably captured. In particular, the Purkinje reflex can be reliably generated. By the form of configuration of the illumination ring, the illumination point or illumination half ring or by means of illumination sectors, torsions can for example also be recognized, since relative position variations of the illumination device to the Purkinje image can also be captured besides the illumination. Thereby, it is possible that the Purkinje image can be captured by means of different illumination devices.

It is further advantageous that the patient interface is illuminated with infrared light by means of an infrared illumination device and the optical capturing device is configured such that infrared light reflected on the patient interface at least in certain areas is captured. In particular, the infrared light is light, which is not perceivable by the patient. Thereby, it is allowed that the position of the patient interface can be reliably captured in an approaching procedure and yet the patient is not unnecessarily impaired.

It is further advantageous if the patient interface is configured electrically insulated and/or sterile. Thereby, an electrical voltage is in particular prevented from transitioning to the eye for example in a docking procedure of the patient interface to the eye. Further, if the patient interface is formed sterile, germs can be prevented from being transferred from the patient interface to the eye.

According to a further advantageous form of configuration, with a deviation above a preset deviation threshold value, the patient interface is newly positioned as the control signal. In other words, if the deviation should be correspondingly high, it can be provided that a docking procedure between the patient interface and the eye then is not performed, but that the patient interface is newly positioned and newly approached to the eye.

It is also advantageous if a position variation of the eye surgical laser, in particular of the laser beam, is performed with a deviation above a preset deviation threshold value. For example, if the deviation should be correspondingly high, thus, the eye surgical laser can be newly positioned. For example, a displacement of the beam deflection device can be performed. Thereby, it is allowed that the treatment on the patient can nevertheless be reliably performed even with a deviation.

Further, it has proven advantageous if the Purkinje image is captured by means of a capturing device arranged at the patient interface. In other words, the capturing device can be arranged at the patient interface. Thereby, it is allowed that the capturing device can be simultaneously and in particular reliably moved with the patient interface, whereby the Purkinje image can be reliably captured during the treatment.

A further aspect of the invention relates to a treatment apparatus with at least one eye surgical laser for the separation of a volume body with predefined interfaces of a human or animal eye for example by means of photodisruption and with at least one control device for the laser or lasers, which is formed to execute the steps of the method according to the preceding aspect.

Therein, the laser is suitable to emit laser pulses in a wavelength range between 300 nm and 1,400 nm, preferably between 700 nm and 1,200 nm, at a respective pulse duration between 1 fs and 1 ns, preferably between 10 fs and 10 ps, and a repetition frequency of greater than 10 kHz, preferably between 100 kHz and 100 MHz.

In an advantageous form of configuration of the treatment apparatus, the treatment apparatus comprises a storage device for at least temporarily storing at least one control dataset, wherein the control dataset or datasets include(s) control data for positioning and/or focusing individual laser pulses in the cornea, and includes at least one beam device for beam guidance and/or beam shaping and/or beam deflection and/or beam focusing of a laser beam of the laser. Therein, the mentioned control datasets are usually generated based on a measured topography and/or tachymetry and/or morphology of the cornea to be treated and the type of the pathologically and/or unnaturally altered area to be removed within the cornea.

Further features and the advantages thereof can be taken from the descriptions of the first inventive aspect, wherein advantageous configurations of each inventive aspect are to be regarded as advantageous configurations of the respectively other inventive aspect.

A third aspect of the invention relates to a computer program including commands, which cause the treatment apparatus according to the second inventive aspect to execute the method steps according to the first inventive aspect. A fourth aspect of the invention relates to a computer-readable medium, on which the computer program according to the third inventive aspect is stored. Further features and the advantages thereof can be taken from the descriptions of the first and the second inventive aspect, wherein advantageous configurations of each inventive aspect are to be regarded as advantageous configurations of the respectively other inventive aspect.

Further features are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

The figures show the following.

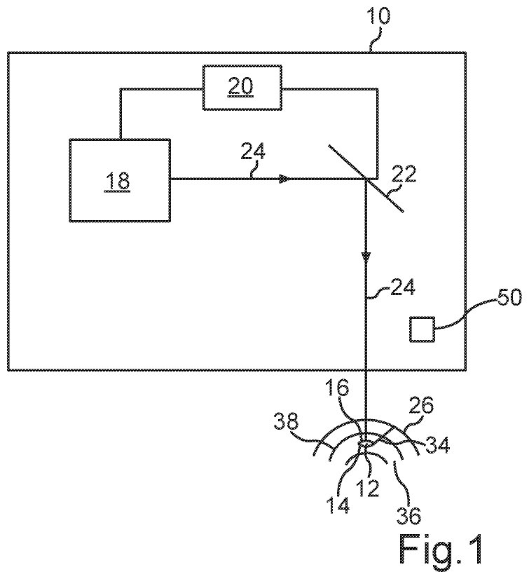

FIG. 1 is a schematic representation of a treatment apparatus according to the invention.

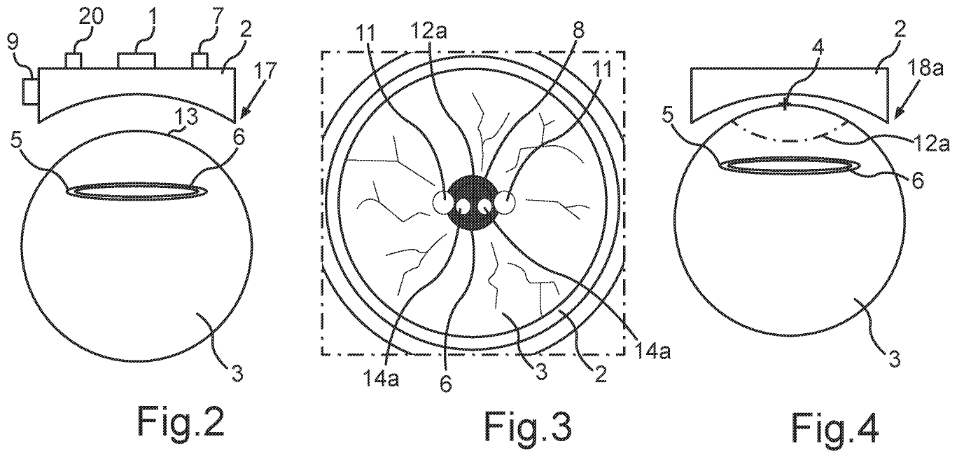

FIG. 2 is a schematic side view of an embodiment of a treatment apparatus with a patient interface in a first situation.

FIG. 3 is a schematic top view to an eye of a patient.

FIG. 4 is a further schematic side view to an embodiment of the patient interface in a further situation.

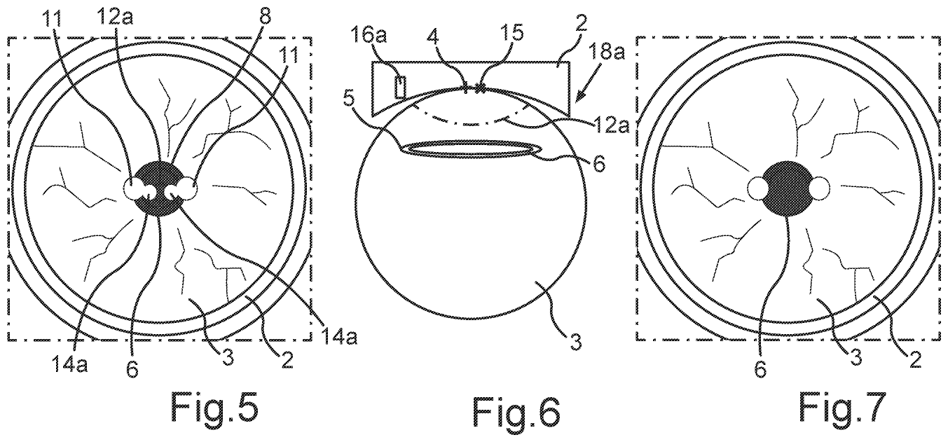

FIG. 5 is a further schematic top view to an eye of a patient.

FIG. 6 is a further schematic side view to an embodiment of the patient interface in a further situation.

FIG. 7 is a further schematic top view to an eye of a patient in a further situation.

In the figures, identical or functionally identical elements are provided with the same reference characters.

FIG. 1 shows a schematic representation of a treatment apparatus 10 with an eye surgical laser 18 for the separation of a predefined corneal volume or volume body 12 with for example predefined interfaces 14, 16 of a cornea 13 (FIG. 2) of a human or animal eye 3 (FIG. 2) for example by means of photodisruption. One recognizes that a control device 20 for the laser 18 is formed besides the laser 18, such that it emits pulsed laser pulses in a predefined pattern into the cornea 13 in the present embodiment, wherein the interfaces 14, 16 of the volume body 12 to be separated are generated by the predefined pattern by means of photodisruption. In the illustrated embodiment, the interfaces 14, 16 form a lenticular volume body 12, wherein the position of the volume body 12 is selected in this embodiment such that a pathological and/or unnaturally altered area within a stroma 36 of the cornea 13 is enclosed. Furthermore, it is apparent from FIG. 1 that the so-called Bowman's membrane 38 is formed between the stroma 36 and an epithelium 28.

Furthermore, one recognizes that the laser beam 24 generated by the laser 18 is deflected towards a surface 26 of the cornea by means of a beam deflection device 22 such as for example a scanner. The beam deflection device 22 is also controlled by the control device 20 to generate the mentioned predefined pattern in the cornea. The beam deflection device 22 for example comprises two mirrors. The incident laser beam 24 can be rotated by rotation around a rotational axis. In a neutral pose of the mirrors, a so-called optical axis 4 (FIG. 2) of the laser beam 24 is in particular formed.

The illustrated laser 18 is a photodisruptive laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. Alternatively to the treatment apparatus 10 shown in FIG. 1, a method for ablative removal of the volume body 12 can also be used.

In addition, the control device 20 comprises a storage device (not illustrated) for at least temporarily storing at least one control dataset, wherein the control dataset or datasets include(s) control data for positioning and/or for focusing individual laser pulses in the cornea 13. The position data and/or focusing data of the individual laser pulses are generated based on a previously measured topography and/or pachymetry and/or the morphology of the cornea and the pathological and/or unnaturally altered area 32 for example to be removed within the stroma 36 of the eye.

FIG. 2 purely exemplarily shows the treatment apparatus 10 in a first situation in a further schematic side view. The treatment apparatus 10 comprises a patient interface 2. The patient interface 2 is formed for the eye surgical laser 18 of the treatment apparatus 10 for the eye 3 of the patient not illustrated. The patient interface 2 can be coupled to the treatment apparatus 10 for example by means of a connection device 1 for guiding the patient interface 2.

In FIG. 2, it is shown that the patient interface 2 can for example have a distance to the eye 3 of for example 5 cm in the illustrated situation. Further, an iris 5 as well as a pupil 6 is shown at the eye 3 in FIG. 2.

In particular, a current pupil position 17 of the eye 3 can be additionally captured by means of the optical capturing device 9 for determining the current position.

FIG. 3 shows a view through the patient interface 2 to the eye 3 of the patient in a top view. In particular, the pupil 6 is shown hatched.

In the method for determining a current position of the patient interface 2, a target position of the patient interface 2 relative to the optical axis 4 of the laser beam 24 in the neutral pose of the beam deflection device 22 is determined. The patient interface 2 is positioned in a preset area in front of the optical axis 4. Illuminating the patient interface 2 by means of an illumination device 7 of the treatment apparatus 10 is effected. A Purkinje image 8, which is associated with the patient interface 2, is captured by means of an optical capturing device 9 of the treatment apparatus 10. Comparing the captured Purkinje image 8 to the optical axis 4 and determining the current position of the patient interface 2 depending thereon are effected. The current position is compared to the target position and with a deviation of the current position from the target position, a control signal is output to a control device 20 of the treatment apparatus 10.

In particular, FIG. 3 further shows that a static projection 11, in particular two static projections 11, are generated by the treatment apparatus 10, in particular by the illumination device 7, on the anterior surface of the patient interface 2. Further, the Purkinje image 8 is shown as a ring on the pupil 6, which is a static projection of the illumination device 7 on the anterior surface of the patient interface 2. Further, a dynamic projection ring 12a is shown, which is generated on an anterior surface of the cornea 13 of the eye 3, and can for example also be a Purkinje reflex on the cornea 13. Further, two dynamic projections 14a of the illumination device 7 are shown, which are generated on the anterior surface of the cornea 13.

In particular, it can be provided that a first order or second order Purkinje reflex is captured as the Purkinje image 8. Presently, a first order Purkinje reflex is in particular shown.

Further, the optical capturing device 9 can in particular be formed as a camera.

Further, it can be provided that with an ascertained deviation, a control signal is generated such that a position correction of the patient interface 2 or of the optical axis 4 is performed. Furthermore, it can be provided that after an ascertained deviation below a preset deviation threshold value, a control signal is generated such that a docking procedure of the patient interface 2 to the eye 3 is performed. The patient interface 2 can also be illuminated by means of an illumination ring or illumination point or by means of an illumination half ring or by means of illumination sectors as the illumination device 7 for generating the Purkinje image 8. Similarly, it can be provided that the patient interface 2 is illuminated with infrared light by means of an infrared illumination device as the illumination device 7 and the optical capturing device 9 is configured such that infrared light reflected on the patient interface 2 at least in certain areas is captured.

FIG. 4 shows the patient interface 2 in an approached state in a schematic side view, thus during an approaching procedure 18a to the eye 3. For example, as presently, a distance of the patient interface 2 to the eye 3 can be 2 to 3 mm. In particular, a Purkinje image 8 is shown on the eye 3. In particular, FIG. 4 shows the optical axis 4. For example, if a deviation above a preset deviation threshold value should now be ascertained, thus, the patient interface 2 can be newly positioned. Further, it can be provided that with a deviation above a preset deviation threshold value, a position variation of the eye surgical laser 18, in particular of the laser beam 24, is performed in that the beam deflection device 22 is for example positioned.

FIG. 5 shows the eye 3 in a further top view. In FIG. 5, the position of the patient interface 2 is in particular as it is illustrated in the side view in FIG. 4. In FIG. 5, a corresponding displacement and thus a deviation of the current position from the target position can in particular be registered. Based on this displacement, the control signal for the treatment apparatus 10, in particular for controlling the patient interface 2, can now be generated.

FIG. 6 shows the eye 3 in a schematic side view during the docking procedure of the patient interface 2. In particular, the patient interface 2 docks to a cornea apex 15 of the eye 3. In particular, the cornea apex 15 is displaced to the optical axis 4. In FIG. 6, it is in particular shown that the patient interface 2 is configured electrically insulated and/or sterile such that electrical voltages cannot transition from the patient interface 2 to the eye 3. In particular, germs either cannot be transferred from the patient interface 2 to the eye 3.

Further, FIG. 6 shows that the eye 3 is sucked onto and fixed to the patient interface 2 by means of a suction device 16a of the patient interface 2 after the docking procedure.

FIG. 7 shows the eye 3 with the patient interface 2 in the top view according to the side view of FIG. 6 in a schematic top view, wherein the Purkinje image 8 herein has nearly disappeared due to docking to the eye 3.

1.-14. (canceled)

15. A method for determining a current position of a patient interface of an eye surgical laser of a treatment apparatus for an eye of a patient relative to an optical axis of a laser beam of the laser in a neutral pose of a beam deflection device of the treatment apparatus, comprising the steps of:
determining a target position of the patient interface relative to the optical axis;
positioning the patient interface in a preset area in front of the optical axis;
illuminating the patient interface by means of an illumination device of the treatment apparatus;
capturing a Purkinje image, which is associated with the patient interface, by means of an optical capturing device of the treatment apparatus;
comparing the captured Purkinje image to the optical axis and determining the current position of the patient interface depending thereon; and
comparing the current position to the target position and with a deviation of the current position from the target position, outputting a control signal to a control device of the treatment apparatus.

16. The method according to claim 15, wherein a first order or second order Purkinje reflex is captured as the Purkinje image.

17. The method according to claim 15, wherein with an ascertained deviation, a control signal is generated such that a position correction of the patient interface or of the optical axis is performed.

18. The method according to claim 15, wherein after an ascertained deviation below a preset deviation threshold value, a control signal is generated such that a docking procedure of the patient interface to the eye is performed.

19. The method according to claim 15, wherein said patient interface is illuminated by means of an illumination ring or illumination point or by means of an illumination half ring or by means of illumination sectors of the illumination device for generating the Purkinje image.

20. The method according to claim 15, wherein said patient interface is illuminated with infrared light by means of an infrared illumination device and the optical capturing device is configured such that infrared light reflected on the patient interface at least in certain areas is captured.

21. The method according to claim 15, wherein the patient interface is configured electrically insulated and/or sterile.

22. The method according to claim 15, wherein with a deviation above a preset deviation threshold value, the patient interface is newly positioned as the control signal.

23. The method according to claim 15, wherein with a deviation above a preset deviation threshold value, a position variation of the eye surgical laser, in particular of the laser beam, is performed.

24. The method according to claim 15, wherein the Purkinje image is captured by means of an optical capturing device arranged at the patient interface.

25. A treatment apparatus with at least one surgical laser for the separation of a volume body of a human or animal eye, with at least one control device for the laser or lasers, and with a patient interface for docking to the eye, which is designed to perform the following method steps:
determining a target position of the patient interface relative to the optical axis;
positioning the patient interface in a preset area in front of the optical axis;
illuminating the patient interface by means of an illumination device of the treatment apparatus;
capturing a Purkinje image, which is associated with the patient interface, by means of an optical capturing device of the treatment apparatus;
comparing the captured Purkinje image to the optical axis and determining the current position of the patient interface depending thereon; and
comparing the current position to the target position and with a deviation of the current position from the target position, outputting a control signal to a control device of the treatment apparatus.

26. The treatment apparatus according to claim 25, wherein a first order or second order Purkinje reflex is captured as the Purkinje image.

27. The treatment apparatus according to claim 25, wherein with an ascertained deviation, a control signal is generated such that a position correction of the patient interface or of the optical axis is performed.

28. The treatment apparatus according to claim 25, wherein after an ascertained deviation below a preset deviation threshold value, a control signal is generated such that a docking procedure of the patient interface to the eye is performed.

29. The treatment apparatus according to claim 25, wherein said patient interface is illuminated by means of an illumination ring or illumination point or by means of an illumination half ring or by means of illumination sectors of the illumination device for generating the Purkinje image.

30. The treatment apparatus according to claim 25, wherein said patient interface is illuminated with infrared light by means of an infrared illumination device and the optical capturing device is configured such that infrared light reflected on the patient interface at least in certain areas is captured.

31. The treatment apparatus according to claim 25, wherein the patient interface is configured electrically insulated and/or sterile.

32. The treatment apparatus according to claim 25, wherein with a deviation above a preset deviation threshold value, the patient interface is newly positioned as the control signal.

33. The treatment apparatus according to claim 25, wherein with a deviation above a preset deviation threshold value, a position variation of the eye surgical laser, in particular of the laser beam, is performed.

34. The treatment apparatus according to claim 25, wherein the Purkinje image is captured by means of an optical capturing device arranged at the patient interface.

35. The treatment apparatus according to claim 25, wherein the control device comprises at least one storage device for at least temporarily storing at least one control dataset, wherein the control dataset or datasets include(s) control data for positioning and/or for focusing individual laser pulses in the cornea, and where-in the control dataset or datasets include(s) control data for positioning the patient interface; and includes at least one beam deflection device for beam guidance and/or beam shaping and/or beam deflection and/or beam focusing of a laser beam of the laser.

36. A computer program including commands that cause the treatment apparatus according to claim 25 to execute the method steps described therein.

37. A computer-readable medium having instructions stored thereon that, when executed by a processor, cause the processor to determine a current position of a patient interface of an eye surgical laser of a treatment apparatus for an eye of a patient relative to an optical axis of a laser beam of the laser in a neutral pose of a beam deflection device of the treatment apparatus, the processor:
determining a target position of the patient interface relative to the optical axis;
positioning the patient interface in a preset area in front of the optical axis;
illuminating the patient interface by means of an illumination device of the treatment apparatus;
capturing a Purkinje image, which is associated with the patient interface, by means of an optical capturing device of the treatment apparatus;
comparing the captured Purkinje image to the optical axis and determining the current position of the patient interface depending thereon; and
comparing the current position to the target position and with a deviation of the current position from the target position, outputting a control signal to a control device of the treatment apparatus.

38. The computer-readable medium of claim 37, wherein a first order or second order Purkinje reflex is captured as the Purkinje image.

39. The computer-readable medium of claim 37, wherein with an ascertained deviation, a control signal is generated such that a position correction of the patient interface or of the optical axis is performed.

40. The computer-readable medium of claim 37, wherein after an ascertained deviation below a preset deviation threshold value, a control signal is generated such that a docking procedure of the patient interface to the eye is performed.

41. The computer-readable medium of claim 37, wherein said patient interface is illuminated by means of an illumination ring or illumination point or by means of an illumination half ring or by means of illumination sectors of the illumination device for generating the Purkinje image.

42. The computer-readable medium of claim 37, wherein said patient interface is illuminated with infrared light by means of an infrared illumination device and the optical capturing device is configured such that infrared light reflected on the patient interface at least in certain areas is captured.

43. The computer-readable medium of claim 37, wherein the patient interface is configured electrically insulated and/or sterile.

44. The computer-readable medium of claim 37, wherein with a deviation above a preset deviation threshold value, the patient interface is newly positioned as the control signal.

45. The computer-readable medium of claim 37, wherein with a deviation above a preset deviation threshold value, a position variation of the eye surgical laser, in particular of the laser beam, is performed.

46. The computer-readable medium of claim 37, wherein the Purkinje image is captured by means of an optical capturing device arranged at the patient interface.
